(19) United States
(12) Patent Application Publication
(10) Pub. No.: US 2006/0247597 A1
(43) Pub. Date: Nov. 2, 2006

(54) Garment with a reversible fastening system

(75) Inventors: Cynthia L. Hogan, Hortonville, WI (US); David L. Zenker, Neenah, WI (US)

Correspondence Address: KIMBERLY-CLARK WORLDWIDE, INC., 401 NORTH LAKE STREET, NEENAH, WI, 54956, US

(73) Assignee: Kimberly-Clark Worldwide, Inc.

(21) Appl. No.: 11/117,902

(22) Filed: Apr. 29, 2005

(51) Int. Cl. A61F 13/15

(52) U.S. Cl. 604/391

(57) ABSTRACT

A reversible fastening system allows an absorbent article to be fastened in a first condition, with a pair of first fasteners engaging the garment facing surface of a first waist region. Alternatively, the absorbent article may be fastened in a second condition, with a pair of second fasteners engaging the garment facing surface of a second waist region. The first condition can be generally viewed as the “reverse” of the second condition.

### BACKGROUND OF THE INVENTION

The present invention relates to disposable absorbent articles for absorbing body fluids and exudates, such as urine and fecal material. More particularly, the present invention relates to absorbent articles, such as disposable diapers and adult incontinence garments, which include a reversible fastening system.

Absorbent articles are typically comprised of at least three general layers. These include an absorbent core placed between a liquid permeable inner liner and a liquid impermeable outer cover. The inner liner and outer cover can comprise one or more individual layers of materials, and additional layers can also be interposed between any of the general layers. For example, in a disposable diaper, the inner liner can comprise a surge layer consisting of thermoplastic fibers positioned beneath a thermoplastic mesh. Further, many disposable articles contain a pair of fasteners attached to the rear of the article which then fasten to the front of the article to secure the article to the body of the wearer. Care givers have become accustomed to this manner of donning absorbent articles and may be reluctant to try alternative donning methods, such as fastening from front to back.

Many absorbent cores within absorbent articles are asymmetric about the lateral centerline; the cores are designed with more absorbent capacity in either the front or back of the diaper. With conventional fastening systems, the higher absorbent capacity can not be adjusted from front to back. A user spending a large amount of time on their back may necessitate a higher amount of absorbent capacity in the back of the absorbent article. Likewise, a user spending a large amount of time on their stomach may necessitate a higher amount of absorbent capacity in the front of the absorbent article. Conventional absorbent articles have not provided an adjustability of absorbency utilizing a consistent donning method.

Therefore, there remains a need for a reversible disposable diaper being able to fasten from back to front.

### SUMMARY

In response to the foregoing need, the present inventors undertook intensive research and development efforts that resulted in the discovery of an absorbent article including a reversible fastening system securing the absorbent article about the waist of the wearer. The reversible fastening systems allows the absorbent articles of the present invention to be fastened in a first condition, with a pair of first fasteners engaging the garment facing surface of a first waist region. Alternatively, the absorbent articles of the present invention may be fastened in a second condition, with a pair of second fasteners engaging the garment facing surface of a second waist region. The first condition can be generally viewed as the “reverse” of the second condition.

One version of the present invention includes a disposable absorbent article having opposed longitudinal side edges, opposed lateral end edges, a first waist region, a second waist region and a crotch region which extends between and connects the first waist region and the second waist region. The disposable absorbent article includes a bodyfacing surface, a garment facing surface an absorbent core and a reversible fastening system. The reversible fastening system includes a pair of first fasteners, at least a portion of each first fastener being situated inboard from each longitudinal side edge of the second waist region, each first fastener being configured to engage at least a portion of the garment facing surface of the first waist region. The reversible fastening system also includes a pair of second fasteners, at least a portion of each second fastener being situated inboard from each longitudinal side edge of the first waist region, each second fastener being configured to engage at least a portion of the garment facing surface of the second waist region. Further, with the lateral end edge in the first waist region and the lateral end edge in the second waist region aligned, the pair of first fasteners and the pair of second fasteners longitudinally overlap another.

Another version of the present invention provides a disposable absorbent article having opposed longitudinal side edges, opposed lateral end edges, a lateral centerline, a first waist region, a second waist region and a crotch region which extends between and connects the first waist region and the second waist region. The disposable absorbent article includes a bodyfacing surface, a garment facing surface, an absorbent core and a reversible fastening system. The reversible fastening system includes a pair of first fasteners, at least a portion of each first fastener being situated inboard from each longitudinal side edge of the second waist region, each first fastener being configured to engage at least a portion of the garment facing surface of the first waist region. The reversible fastening system also includes a pair of second fasteners, at least a portion of each second fastener being situated inboard from each longitudinal side edge of the first waist region, each second fastener being configured to engage at least a portion of the garment facing surface of the second waist region. Further, the first fasteners are inoperable when the second fasteners are engaged with the garment facing surface in the first waist region.

Still another version of the present invention provides a method of providing a system to diaper a child including providing the disposable absorbent article as described above. The method also includes providing instructions to a care give to don the disposable absorbent article in one of two conditions and enabling the wearer to successfully apply the disposable absorbent article by following the instructions.

### DRAWINGS

The foregoing and other features and aspects of the present invention and the manner of attaining them will become more apparent, and the invention itself will be better understood by reference to the following description, appended claims and accompanying drawings, where:

FIG. 1 illustrates a plan view of the bodyfacing surface of a representative disposable absorbent article incorporating a version of the reversible fastening system;

FIG. 2 illustrates a plan view of the garment facing surface of a representative disposable absorbent article incorporating a second version of the reversible fastening system;

FIG. 3 illustrates a plan view of the bodyfacing surface of a further representative disposable absorbent article incorporating a version of the reversible fastening system;

FIG. 4 representatively shows a perspective view of an example of a disposable absorbent article (an infant diaper) incorporating a version of the reversible fastening system fastened in a first condition;

FIG. 4A representatively shows a perspective view of the disposable absorbent article of FIG. 4 fastened in a second condition; and

FIG. 5 illustrates a version of an ear suitable for incorporation into a version of the reversible fastening system.

### DESCRIPTION

The present disclosure of the invention will be expressed in terms of its various components, elements, constructions, configurations, arrangements and other features that may also be individually or collectively be referenced by the term, “aspect(s)” of the invention, or other similar terms. It is contemplated that the various forms of the disclosed invention may incorporate one or more of its various features and aspects, and that such features and aspects may be employed in any desired operative combination thereof.

It should also be noted that, when employed in the present disclosure, the terms “comprises”, “comprising” and other derivatives from the root term “comprise” are intended to be open-ended terms that specify the presence of any stated features, elements, integers, steps, or components, and are not intended to preclude the presence or addition of one or more other features, elements, integers, steps, components, or groups thereof.

As used herein, the term “prefastened” refers to a condition wherein the absorbent article has a fastening feature, which is engaged or fastened prior to use by the wearer. For example, the fastening feature of the absorbent article may be engaged or fastened during the manufacturing process.

The present invention is directed to disposable garments having a reversible fastening system. Additionally, the present invention is directed to a garment having a unique combination of features that provide previously unrecognized and unexpected benefits. This detailed description of the present invention will include a description of a representative disposable garment including the various components of such garments. The description of the representative disposable garment will also include a description of many features encompassed by the present invention.

Representative Disposable Garment

The present invention concerns a reversible fastening system for use with disposable garments. The disposable garments are adapted to be worn adjacent to the body of a wearer, that is, a disposable garment that is similar to a disposable diaper. It is understood that the features of the present invention are equally adaptable for other types of disposable garments such as diaper-pants, training pants, feminine hygiene garments and adult incontinence garments.

As used herein, the term “disposable” refers to garments which are intended to be discarded after a limited use and which are not intended to be laundered or otherwise restored for reuse. The disposable garments of the present invention will be described in terms of a disposable diaper which is adapted to be worn by infants about the lower torso.

With regard to the designated surfaces of a disposable absorbent article and its components, the various upper or bodyfacing surfaces are configured to face toward the body of the wearer when the disposable absorbent article is worn by the wearer for ordinary use. The various opposing, lower or garment facing surfaces are configured to face away from the wearer's body when the disposable absorbent article is worn by the wearer.

As used herein, reference to two materials or elements being “joined” is intended to refer to the situation wherein the two materials or elements are directly joined to one another or where they are indirectly joined to one another or where they are indirectly joined to an intermediate element. Similarly, methods of joining two materials or elements include forming the elements or materials integrally, or attaching the elements together such as through the use of adhesive bonds, sonic bonds, thermal bonds, pinning, stitching, or a variety of other attachment techniques known in the art, as well as combinations thereof.

Stretchable materials may include materials that are extensible and materials that are elastomeric. Extensible materials typically have lower capacities to retract to their original lengths after stretching, while elastomeric materials typically have a greater range of stretch and come close to completely retracting to their original lengths.

It should be noted that the elongation, extension or permanent deformation properties of an extensible material are determined when the material is dry. Additionally, the percentage of elongation, extension or permanent deformation can be determined in accordance with the following formula:
100*[(L−Lo)/(Lo)]
where: L=elongated length; and
   Lo=initial length.

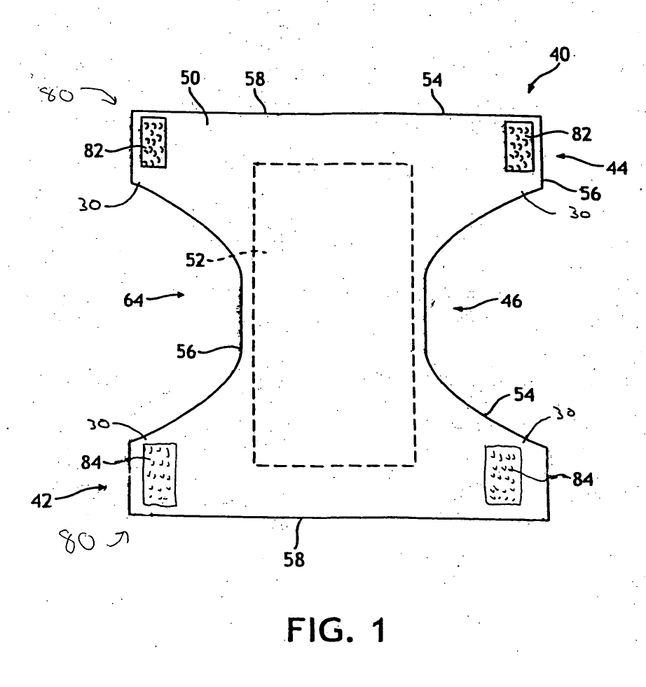

FIGS. 1-4 representatively illustrate examples of disposable garments, in this instance a diaper (as generally indicated at 40). Referring to FIGS. 1-4, the diapers (40) define a first waist region (42), a second waist region (44) and a crotch region (46) which extends between and connects the first (42) and second (44) waist regions. The crotch region (46) of the diaper (40) comprises the portion of the diaper which, when worn, is positioned between the legs of the wearer and covers the lower torso of the wearer.

The diaper (40) also includes an outer cover (48), a bodyside liner (50), and an absorbent core (52) situated between the outer cover (48) and the liner (50). The outer edges of the diaper (40) define a periphery (54) with laterally opposed, longitudinally extending side edges (56) and longitudinally opposed, laterally extending end edges (58). The diaper (40) may also include a system of elastomeric gathering members, such as leg elastics and waist elastics (not illustrated). The longitudinal side edges (56) define leg openings (64) for the diaper (40), and optionally are curvilinear and contoured. The lateral end edges (58) are illustrated as straight, but optionally, may be curvilinear. The diaper (40) additionally has a longitudinal centerline (66) and a lateral centerline (68). The diaper (40) may also include additional components to assist in the acquisition, distribution and storage of bodily waste. For example, the diaper (40) may include a transport layer, such as described in U.S. Pat. No. 4,798,603, issued to Meyer et al., or a surge management layer, such as described in European Patent Application Publication No. 0 539 703, published May 5, 1993. These layers may be placed symmetrically about the lateral centerline (68); alternatively these layers may be placed asymmetrically about the lateral centerline (68).

As used herein, the term “inboard” is intended to refer to the direction from an edge toward a respective centerline. The term “outboard” is intended to refer to a direction away from a respective centerline.

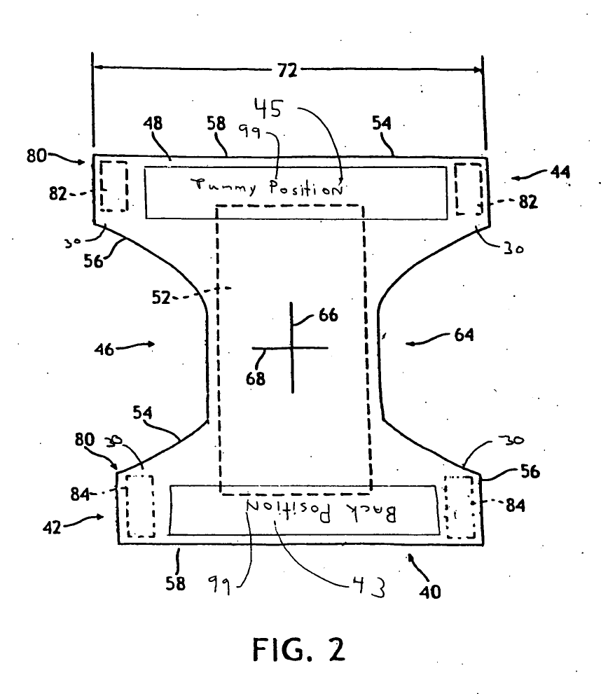

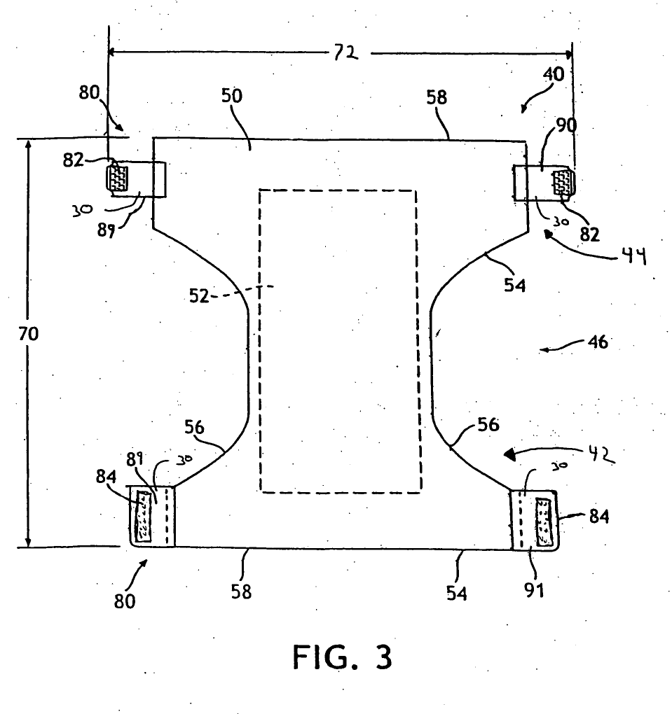

The diaper (40) generally defines a longitudinally extending length dimension (70), and a laterally extending width dimension (72) (as representatively illustrated in FIGS. 2 and 3).

The diaper may have any desired shape, such as rectangular, I-shaped, an hourglass shape, or a T-shape.

The outer cover (48) and the liner (50) may be coextensive (e.g., FIG. 2), or optionally, may be non-coextensive. Either or both of the outer cover (48) and the liner (50) may have length and width dimensions which are generally larger than those of the absorbent core (52) and extend beyond the corresponding dimensions of the absorbent core (52) to provide longitudinal side edges (56) and lateral end edges (58) which may be connected or otherwise associated together in an operable manner.

The outer cover (48) can be composed of various materials and is suitably liquid impermeable. Desirably the outer cover (48) is made of a stretchable material. In a particular aspect, the outer cover (48) is made of an elastomeric material. Suitable elastomeric materials are stretchable in one or more directions. Elastomeric materials may include cast or blown films, foams, or meltblown fabrics composed of polyethylene, polypropylene, or polyolefin copolymers, as well as combinations thereof. The elastomeric materials may include PEBAX elastomer (available from AtoChem located in Philadelphia, Pa.), HYTREL elastomeric polyester (available from E.I. DuPont de Nemours of Wilmington, Del.), KRATON elastomer (available from Kraton Polymers of Houston, Tex.), or strands of LYCRA elastomer (available from E.I. DuPont de Nemours of Wilmington, Del.), or the like, as well as combinations thereof. The outer cover (48) may include materials that have elastomeric properties through a mechanical process, printing process, heating process, or chemical treatment. For example, such materials may be apertured, creped, neck-stretched, heat activated, embossed, and micro-strained; and may be in the form of films, webs, and laminates.

In particular aspects, the outer cover (48) may include a 0.4 ounces per square yard (osy) (13.6 grams per square meter (gsm)) basis weight layer of G2760 KRATON elastomer strands adhesively laminated with a 0.3 gsm layer of adhesive between two facings. Each facing can be composed of a thermal point bonded bicomponent spunbond non-woven fibrous web having a 0.7 osy (23.7 gsm) basis weight. The adhesive, designated as H2525A., is an adhesive which is supplied by AtoFindley Adhesive, a business having offices in Wauwatosa, Wis. The elastomer strands are placed and distributed to provide approximately 12 strands of KRATON elastomer per inch (2.54 cm) of lateral width of the outer cover (48).

Materials suitable for a biaxially stretchable outer cover (48) include biaxially stretchable materials and biaxially elastic stretchable materials. One example of a suitable outer cover material can include a 0.3 osy (10 gsm) polypropylene spunbond that is necked 60% in the lateral direction and creped 60% in the longitudinal direction, laminated with 3 grams per square meter (gsm) AtoFindley Adhesives H2525A styrene-isoprene-styrene based adhesive to 8 gsm PEBAX 2533 film with 20% TiO₂ concentrate. The outer cover (48) can suitably be stretched, laterally and/or longitudinally, by at least 30% (to at least 130% of an initial (unstretched) width and/or length of the outer cover (48)). More suitably, the outer cover (48) can be stretched laterally and/or longitudinally, by at least 50% (to at least 150% of the unstretched width or length of the outer cover (48)). Even more suitably, the outer cover (48) can be stretched, laterally and/or longitudinally, by at least 100% (to at least 200% of the unstretched width or length of the outer cover (48)). Tension force in the outer cover (48) at 50% extension is suitably between 50 and 1000 grams, more suitably between 100 and 600 grams, as measured on a 3 inch (7.62 cm) wide piece of the outer cover material.

Another example of a suitable material for a biaxially stretchable outer cover (48) is a breathable elastic film/nonwoven laminate, described in U.S. Pat. No. 5,883,028, issued to Morman et al, incorporated herein by reference to the extent that it is consistent (i.e., not in conflict) herewith. Examples of materials having two-way stretchability and retractability are disclosed in U.S. Pat. No. 5,116,662, issued to Morman, and U.S. Pat. No. 5,114,781, issued to Morman, both of which are hereby incorporated herein by reference to the extent that each is consistent (i.e., not in conflict) herewith. These two patents describe composite elastic materials capable of stretching in at least two directions. The materials have at least one elastic sheet and at least one necked material, or reversibly necked material, joined to the elastic sheet at least at three locations arranged in a nonlinear configuration, so that the necked, or reversibly necked, web is gathered between at least two of those locations.

In an alternative aspect, the outer cover (48) is made of an extensible material. Extensible materials suitable for use as an outer cover (48) can provide an elongation of at least 10; alternatively, at least 20; alternatively, at least 30; or, alternatively, at least 40% when subjected to a tensile force of 30 gmf per inch (per 2.54 cm). Material suitable for use as an outer cover (48) can also provide a substantially permanent deformation of at least 10; alternatively, at least 15; alternatively, at least 20; alternatively, at least 25; or, alternatively, at least 30% when subjected to a tensile force of 50 gmf per inch (per 2.54 cm) and then allowed to relax, after removal of the tensile force, for a period of 1 minute. It should be readily appreciated that the described removal of the applied force results in a zero applied tensile stress and a zero applied tensile force.

The outer cover (48) can be composed of various materials and is suitably liquid impermeable. If extensible, for example, the outer cover (48) can be composed of a necked fabric, a creped fabric, a crimped fiber fabric, an extendable fiber fabric, a bonded-carded fabric, a micro-pleated fabric, polymer films or the like, as well as combinations thereof. The fabrics may be knit, woven or non-woven materials, such as spunbond fabrics. In a particular aspect, the outer cover (48) can be composed of an extensible laminate of two or more layers. For example, the outer cover (48) may be a necked laminate formed from at least one neckable fabric laminated to at least one extendable film material wherein the necked laminate is extensible in at least one direction. The outer cover material (48), if extensible, may otherwise be a laminate formed from at least one necked fabric laminated to at least one extendable film material. In such a configuration, the laminate need not be necked. For purposes of the present description, the term “nonwoven web” refers to a web of fibrous material that is formed without the aid of a textile weaving or knitting process. The term “fabrics” is used to refer to woven, knitted and nonwoven fibrous webs. An example of an extensible material suitable for use as an outer cover (48) is a 60% necked, polypropylene spunbond having a basis weight of about 1.2 osy (41 gsm).

The liner (50) suitably presents a bodyfacing surface which is compliant, soft feeling, and non-irritating to the wearer's skin. Further, the liner (50) may be less hydrophilic than the absorbent core (52), to present a relatively dry surface to the wearer, and is sufficiently porous to be liquid permeable, permitting liquid to readily penetrate through its thickness. A suitable liner (50) may be manufactured from a wide selection of web materials, such as porous foams, reticulated foams, apertured plastic films, natural fibers, synthetic fibers (for example, polyester or polypropylene fibers), or a combination of natural and synthetic fibers. The liner (50) is suitably employed to help isolate the wearer's skin from liquids held in the absorbent core (50).

Desirably the liner (50) is made of a stretchable material. In a particular aspect, the liner (50) is made of an elastomeric material. Suitable elastomeric materials are stretchable in one or more directions. Suitable elastomeric materials for construction of the liner (50) can include elastic strands, LYCRA elastics, cast or blown elastic films, nonwoven elastic webs, meltblown or spunbond elastomeric fibrous webs, as well as combinations thereof. Examples of suitable elastomeric materials include KRATON elastomers, HYTREL elastomers, ESTANE elastomeric polyurethanes (available from B.F. Goodrich and Company of Cleveland, Ohio), or PEBAX elastomers.

As an additional example, in one aspect the liner (50) suitably includes a non-woven, spunbond polypropylene fabric composed of about 2 to 3 denier fibers formed into a web having a basis weight of about 12 gsm which is necked approximately 60%. Strands of about 9 gsm KRATON G2760 elastomer material placed eight strands per inch (2.54 cm) are adhered to the necked spunbond material. The fabric is surface treated with an operative amount of surfactant, such as about 0.6 percent AHCOVEL Base N62 surfactant, available from ICI Americas, a business having offices in Wilmington, Del. The surfactant can be applied by any conventional means, such as spraying, printing, brush coating or the like. Other suitable materials may be extensible biaxially stretchable materials, such as a neck stretched/creped spunbond.

In an alternative aspect, the liner (50) is made of an extensible material. Extensible liner materials (50) can provide an elongation of at least 50% when subjected to a tensile force of 10 gmf per inch (per 2.54 cm). Extensible materials suitable for use as a liner (50) can also provide a substantially permanent deformation of at least 10% when subjected to a tensile force of 50 gmf per inch (per 2.54 cm) and then allowed to relax, after removal of the tensile force, for a period of 1 minute. It should be readily appreciated that the described removal of the applied force results in a zero applied tensile stress and a zero applied tensile force.

A suitable extensible liner (50) may be manufactured from a wide range of materials including, but not limited to knit, woven and nonwoven materials, apertured formed thermoplastic films, apertured plastic films, hydro-formed films, porous foams, reticulated foams, reticulated thermoplastic films, and thermoplastic scrims. Suitable woven and nonwoven materials can include natural fibers (e.g., wood or cotton fibers), synthetic or modified natural fibers (e.g., polymeric fibers, such as polyester, polypropylene fibers, and polyethylene, or polyvinylalcohol, starch base resins, polyurethanes, cellulose esters, nylon, and rayon fibers), or a combination of natural and synthetic fibers. When the extensible liner material (50) includes a nonwoven web, the web may be spunbonded, carded, wet-laid, meltblown, hydroentangled, combinations of the above, or the like. An example of a suitable extensible liner (50) is a 50% necked, polypropylene spunbond having a basis weight of about 0.5 osy.

As previously mentioned, the liner (50) may be treated with a surfactant. This can be accomplished by a variety of techniques known to those skilled in the art. Treating the liner (50) with a surfactant generally renders the liner (50) more hydrophilic. This typically results in liquid penetrating the liner (50) faster than if it were not treated.

The absorbent core (52) may include a matrix of hydrophilic fibers, such as a web of cellulosic fluff, mixed with particles of a high-absorbency material commonly known as superabsorbent material. In a particular version, the absorbent core (52) includes a mixture of superabsorbent hydrogel-forming particles and wood pulp fluff. The wood pulp fluff may be exchanged with synthetic polymeric, meltblown fibers or with a combination of meltblown fibers and natural fibers. The superabsorbent particles may be substantially homogeneously mixed with the hydrophilic fibers or may be non-uniformly mixed.

The absorbent core (52) may have any of a number of shapes. For example, the absorbent core (52) may be rectangular, I-shaped or T-shaped. It is often desired that the absorbent core (52) be narrower in the crotch portion than the first or second waist regions (42, 44). The absorbent core (52) may be shaped and positioned such that it is symmetric about the lateral centerline (68), alternatively, it may be shaped or positioned such that it is asymmetric about the lateral centerline (68). Further, the absorbent capacity of the core (52) may be symmetric about the lateral centerline (68), alternatively, the absorbent capacity of the core (52) may be asymmetric about the lateral centerline (68). For example, the absorbent core (52) may have a greater absorbent capacity on the second waist region (44) side of the lateral centerline (68) than on the first waist region (42) side of the lateral centerline (68).

The high-absorbency material can be selected from natural, synthetic and modified natural polymers and materials. The high-absorbency materials can be inorganic materials, such as silica gels, or organic compounds, such as crosslinked polymers. The term “crosslinked” refers to any means for effectively rendering normally water-soluble materials substantially water insoluble, but swellable. Such means can include, for example, physical entanglement, crystalline domains, covalent bonds, ionic complexes and associations, hydrophilic associations, such as hydrogen bonding, and hydrophobic associations or Van der Waals forces.

Examples of synthetic, polymeric, high-absorbency materials include the alkali metal and ammonium salts of poly(acrylic acid) and poly(methacrylic acid), poly(acrylamides), poly(vinyl ethers), maleic anhydride copolymers with vinyl ethers and alpha-olefins, poly(vinyl pyrolidone), poly(vinyl morpholinone), poly(vinyl alcohol), and mixtures and copolymers thereof. Further polymers suitable for use in the absorbent core include natural and modified natural polymers, such as hydrolyzed acrylonitrile-grafted starch, acrylic acid grafted starch, methyl cellulose, carboxymethyl cellulose, hydroxypropyl cellulose, and the natural gums, such as alginates, xanthum gum, locust bean gum, and the like. Mixtures of natural and wholly or partially synthetic absorbent polymers can also be useful. Processes for preparing synthetic, absorbent gelling polymers are disclosed in U.S. Pat. No. 4,076,663, issued to Masuda et al., and U.S. Pat. No. 4,286,082, issued to Tsubakimoto et al.

The high-absorbency material may be in a variety of geometric forms. It is desired that the high-absorbency material be in the form of discrete particles. However, the high-absorbency material may also be in the form of fibers, flakes, rods, spheres, needles, or the like. Often, the high-absorbency material is present in the absorbent core (52) in an amount of from about 5 to about 100 weight percent based on total weight of the absorbent core (52).

The disposable absorbent articles described herein also include a reversible fastening system (80) for securing the absorbent article about the waist of the wearer. The illustrated versions of the diaper (40) include such a reversible fastening system (80). Specifically, the reversible fastening system includes at least two first fasteners (82) and at least two second fasteners (84). The first fasteners (82) are situated in the second waist region (44) of the diaper (40), located inboard each longitudinal side edge (56) and positioned on the bodyfacing surface of the diaper (40). The first fasteners (82) engage the outer cover (48) of the first waist region (42) of the diaper (40) for holding the diaper on the wearer. Desirably, the first fasteners (82) are releasably engageable directly with the garment facing surface of the outer cover (48). Alternatively, the diaper (40) may include a fastening panel (43) situated in the first waist region (42) of the garment facing surface of the outer cover (48). In this configuration, the fastening panel (43) forms a portion of the garment facing surface of the garment. In such a configuration, the first fasteners (82) are releasably engageable with the fastening panel (43) to maintain the diaper (40) about the waist of the wearer. As representatively illustrated in FIGS. 1, 3 and 4, the first fasteners (82) may be hook type fasteners and the outer cover (48) or fastening panel (43) may be configured to function as a complimentary loop type fastener. Desirably, the first fasteners (82) are hook type fasteners which are releasably engageable with the outer cover (48). Such an arrangement provides the ability to vary the size of the waist opening in very small increments over a wide range to fit the waist of the wearer. The first fasteners (82) may have a variety of shapes and sizes which provide the desired fastening of the diaper (40) about the waist of the wearer.

The reversible fastening system (80) of the present invention further includes at least two second fasteners (84) to provide versatility and flexibility in securing and donning of the diaper (40) about the waist of the wearer. For example, as representatively illustrated in FIGS. 1-4, the diaper (40) may include at least two second fasteners (84) situated in the first waist region (42) of the diaper, located adjacent each longitudinal side edge (56) and positioned on the bodyfacing surface of the diaper (40). The pair of second fasteners (84) and the pair of first fasteners (82) may be positioned substantially symmetrically about the lateral centerline (68); alternatively, they may be positioned asymmetrically about the lateral centerline (68).

The second fasteners (84) engage the garment facing surface of the second waist region (44) of the diaper (40) for holding the diaper on the wearer. Desirably, the second fasteners (84) are releasably engageable directly with the garment facing surface of the outer cover (48). Alternatively, the diaper (40) may include a fastening panel (45) situated in the second waist region (44) of the garment facing surface of the outer cover (48). In this configuration, the fastening panel (45) forms a portion of the garment facing surface of the garment. In such a configuration, the second fasteners (84) are releasably engageable with the fastening panel (45) to maintain the diaper (40) about the waist of the wearer. As representatively illustrated in FIGS. 1, 3 and 4, the second fasteners (84) may be hook type fasteners and the outer cover (48) or fastening panel (45) may be configured to function as a complimentary loop type fastener. Desirably, the second fasteners (84) are hook type fasteners which are releasably engageable with the outer cover (48). Such an arrangement provides the ability to vary the size of the waist opening in very small increments over a wide range to fit the waist of the wearer. The second fasteners (84) may have a variety of shapes and sizes which provide the desired fastening of the diaper (40) about the waist of the wearer.

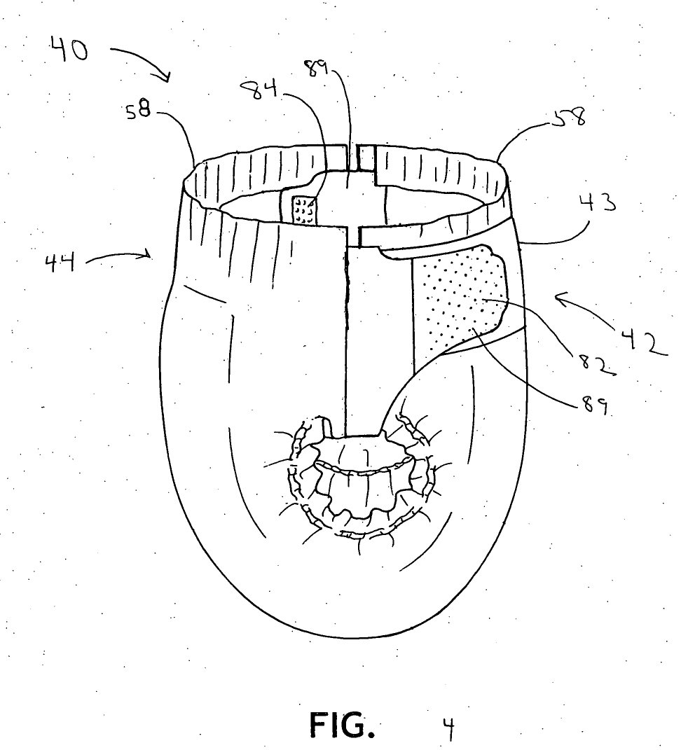

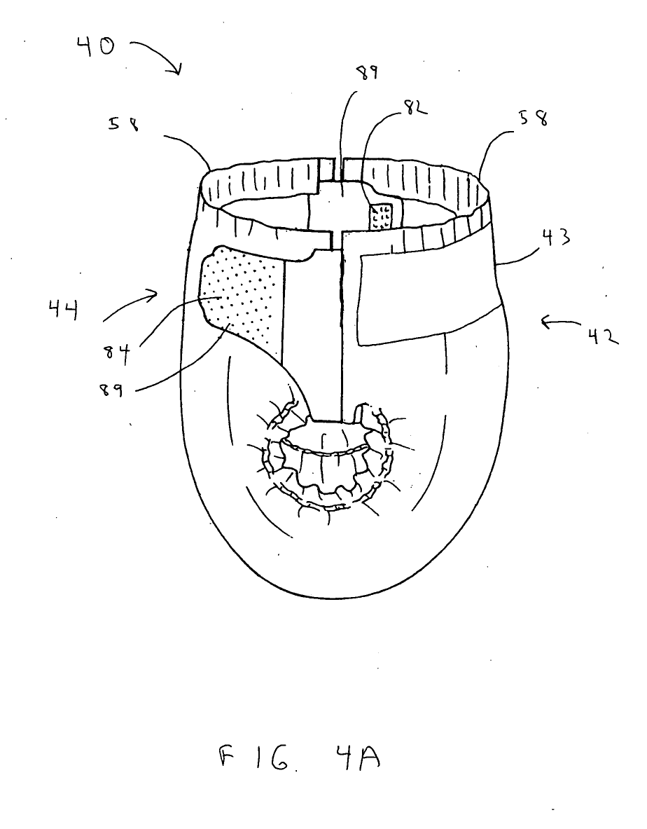

The reversible fastening system (80) may be presented to a user in a flat configuration, such that the user may don the garment without removal of clothes. Alternatively, the reversible fastening system (80) may be presented to a user in a prefastened configuration, such that the user may don the garment as one would don underpants, as shown in FIG. 4.

The term fastening tab as used in the present application refers to the laterally outward portions of the first or second waist region (42, 44). The reversible fastening system (80) includes fastening tabs (30). Fastening tabs (30) may be secured to the bodyfacing surface by ultrasonic bonding at opposing sides of first and second waist regions (42, 44) of the disposable garment (as shown in FIGS. 3 and 4). Each fastening tab (30) includes a bodyfacing surface and a garment facing surface. Each fastening tab (30) further includes first (82) or second (84) fasteners attached to the fastening tabs on the bodyfacing surface.

While ultrasonic bonding is disclosed as a method for mounting fastening tabs (30), other well known methods are contemplated. For example, curing adhesives, stitching, and pressure sensitive adhesives, are all potential mechanisms for suitably and permanently securing the inboard ends of fastening tabs (30). Fastening tabs (30) may be secured to the bodyfacing surface, alternatively, fastening tabs (30) may be secured to the garment facing surface, alternatively, fastening tabs (30) may be secured between the garment facing and bodyfacing surfaces.

Fastening tabs (30) may be formed as an integral part of the garment facing surface and/or the bodyfacing surface (as shown in FIGS. 1 and 2). Such an arrangement reduces the amount of elements needed to form the garment.

Fastening tab (30) may include a tab substrate preferably comprising a non-woven material, such as spunbond-meltblown-spunbond material (SMS). SMS material comprises a layer of meltblown material disposed between and in surface-to-surface relationship with the spunbond layers.

Other materials having suitable characteristics can be substituted for the above-described tab substrates for fastening tab (30). Furthermore, extensible materials can be utilized for the tab substrate.

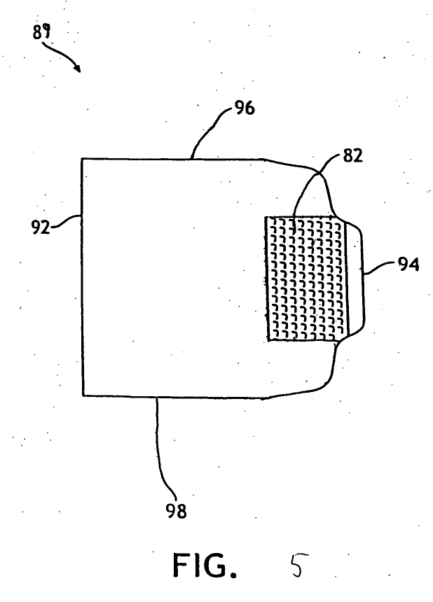

The fastening tab (30) may be provided by one or more ears (89). As illustrated in FIGS. 3, 4 and 5, each ear (89) includes a proximal edge (92), an opposed distal edge (94), a first connecting edge (96), and a second connecting edge (98). As used herein, the proximal edge (92) is that edge of the ear (89) located nearest to the longitudinal centerline (66). The distal edge (94) is that edge of the ear (89) which is opposite the proximal edge (92). The first (96) and second (98) connecting edges connect the proximal edge (92) and the distal edge (94) thereby defining a body of material which at least partially defines an ear (89). The ear (89) may be made of a variety of materials including those that are extensible, non-extensible, elastomeric and/or non-elastomeric. Desirably, the ear (89) is made of a non-elastomeric material.

The diapers (40) illustrated in FIGS. 3 and 4 include a version of the alternative reversible fastening system (80) described herein. Specifically, the reversible fastening system (80) includes at least two first fasteners (82) and at least two second fasteners (84). Typically, the first fasteners (82) are located inboard of a distal edge (94) on a bodyfacing surface of a first ear (90). At least a portion of the proximal edge (92) of the first ear (90) is joined to the second waist region (44) of the diaper (40), with the distal edge (94) of the ear extending outboard from the longitudinal centerline (66) and forming a portion of the side edge (56) of the diaper. Desirably, the first fasteners (82) are releasably engageable directly with the garment facing surface of the outer cover (48). Alternatively, the diaper (40) may include a fastening panel (43) situated in the first waist region (42) of the garment facing surface of the outer cover (48). In such a configuration, the first fasteners (82) are releasably engageable with the fastening panel (43) to maintain the diaper (40) about the waist of the wearer.

The reversible fastening system of this alternate version also includes a pair of second fasteners (84). Specifically, the second fasteners (84) are located inboard of a distal edge (94) on a bodyfacing facing surface of a second ear (91). At least a portion of the proximal edge (92) of the second ear (91) is joined to the first waist region (42) of the diaper (40), with the distal edge (94) of the ear extending outboard from the longitudinal centerline (66) and forming a portion of the side edge (56) of the diaper. Desirably, the second fasteners (84) are hook type fasteners which are releasably engageable directly with the garment facing surface of the outer cover (48). Alternatively, the diaper (40) may include one or more fastening panels (45) on the garment facing surface of the second waist region (44) to which the second fasteners (84) are releasably engageable.

Suitable fasteners are well known to those of skill in the art and can include adhesive tape tab fasteners, hook and loop fasteners, mushroom fasteners, snaps, pins, belts and the like, and combinations thereof. For example, as representatively illustrated in FIGS. 1, 3 and 4, the first fasteners (82) may be hook type fasteners and the outer cover (48) or fastening panel (43) in the first waist region (42) may be configured to function as a complimentary loop type fastener. Desirably, the first fasteners (82) are hook type fasteners which are releasably engageable with at least a portion of an outer cover (48). The second fasteners (84) may also be hook type fasteners (as representatively illustrated in FIGS. 1, 3 and 4). Desirably, the second fasteners (84) are hook type fasteners which are releasably engageable with at least a portion of the outer cover (48) or fastening panel (45) in the second waist region (44). Such an arrangement provides the ability to vary the size of the waist opening in very small increments over a wide range to fit the waist of the wearer. The first (82) and second (84) fasteners may have a variety of shapes and sizes which provide the desired fastening of the diaper about the waist of the wearer.

The absorbent articles of the present invention may be fastened in a first condition, with the pair of first fasteners (82) engaging the garment facing surface of the first waist region (42), as representatively shown in FIG. 4. Alternatively, the absorbent articles of the present invention may be fastened in a second condition, with the pair of second fasteners (84) engaging the garment facing surface of the second waist region (44) (not shown). The first condition can be generally viewed as the “reverse” of the second condition. As shown in FIG. 4, when the lateral end edge (58) in the first waist region (42) and the lateral end edge (58) in the second waist region (44) are aligned, the pair of first fasteners (82) and the pair of second fasteners (84) overlap in the longitudinal direction. More specifically, in the first condition, with the pair of first fasteners (82) engaging the garment facing surface of the first waist region (42), the pair of second fasteners (84) are positioned beneath, or covered by the bodyfacing surface of, the pair of first fasteners (82). In the first condition, the pair of second fasteners (84) are inoperable for their intended purpose. Likewise, in the second condition, as shown in FIG. 4A, with the pair of second fasteners (84) engaging the garment facing surface of the second waist region (44), the pair of first fasteners (82) are positioned beneath, or covered by the bodyfacing surface of, the pair of second fasteners (84). In the second condition, the pair of first fasteners (82) are inoperable for their intended purpose.

Indicia (99) may be included on various portions of the garment and may be configured in various shapes and areas. Indicia (99) may be included on the first (82) or second (84) fasteners. The indicia (99) may appear on the garment facing surface in the first waist region (42) or the second waist region (44), or in the crotch region (46). The indicia (99) may appear on the outer cover (48), alternatively the indicia (99) may appear on one or more fastening panels (43, 45). Indicia (99) may comprise alphanumeric characters (FIG. 2). As used herein and, the term “indicia” is meant to include any type of lines, patterns, ornamental designs, symbols, script, color codes, or other markings which have the capability, either inherently or with additional denotation, to convey information to aid an individual fitting the diaper to the wearer. Indicia may be perceptible using any of the senses.

Visual indicia may include paint, ink, dyes, or other coloring agents applied to, or visible through, a surface, as well as separate elements having indicia, such as a separate sheet of material secured to the surface, colored thread stitched or otherwise secured to the substrate to form the indicia, elastomeric elements having a different color than the substrate and secured thereto, or other elements having substantially the same function and effect, secured to the substrate. “Indicia” also includes luminescent material such as luminescent paint having luminescent pigments that radiate visible light when exposed to ultraviolet light. Examples of suitable luminescent paints are those made with phosphors, such as zinc or cadmium sulfides. Indicia also includes embossing or ultrasonic bonding. For example, embossing can darken the existing color of the material being embossed or provide a texture to the material being embossed. The embossing may reduce the bulk or softness of the material being embossed. Alternatively, in a two layer composite, embossing may allow the color of an underlying layer to become more visible through an upper layer.

Tactile indicia may include embossing, the addition of raised bumps to a surface or other elements which may be felt with the hands to aid an individual fitting the diaper to the wearer

Indicia (99) preferably have bright colors so that the indicia are easily detected by a user. Indicia (99) preferably greatly contrast in color from the (typically white, light pink, or light blue) color of respective personal care articles.

In the embodiment of FIG. 2, the indicia (99) located on the fastening panel (43) in the first waist region (42) include the words “Back Position”. The indicia (99) may be used to indicate a first position with words, graphics or the like. Additionally, the fastening panel (45) in the second waist region (44) may also include indicia (99) to indicate a second position. As shown in FIG. 2, the indicia (99) include the words “Tummy Position”. The second position may also be indicated with words, graphics or the like. The positions may include back, front, prone, supine, toward floor, toward ceiling, or the like. Graphics may also be used to indicate the positions.

Embodiments wherein phosphors are utilized in constructing the indicia have numerous advantages. First, assuming a garment receives ambient light during the day, its indicia containing phosphors may be visible at night or in poor lighting.

Second, such garment will be easier to locate at night due to the luminescent effect of the indicia. Therefore, the garment will be easier to locate without operating a light and awakening other persons. Further, less light would be required for placement on a wearer at night. For example, a spent garment for an infant could be replaced with a fresh garment article having luminescing indicia. The amount of ambient light required to secure such fresh garment would be generally less than the amount of light required if such luminescing material were not available. Therefore, the garment could be changed with, minimal disturbance of the wearing infant or other adults, if present in the same room.

In some embodiments, instructions may be provided to a wearer or caregiver on the proper positioning and use of the present invention. The instructions may include information enabling the wearer to properly don the absorbent article (40) on the wearer in one of two conditions. The instructions may further describe the function and use of the reversible fastening system (80) as well as the improved protection that may result from utilization of one of the two conditions. The instructions may further assist a wearer in understanding and using the indicia (99) for positioning the absorbent article (40). A wearer may reduce leakage by following the instructions provided in combination with use of the absorbent article (40) of the present invention.

Having described the invention in rather full detail, it will be readily apparent that various changes and modifications can be made without departing from the spirit of the invention. All of such changes and modifications are contemplated as being within the scope of the invention as defined by the appended claims and any equivalents thereto.

What is claimed is:

1. A disposable absorbent article having opposed longitudinal side edges, opposed lateral end edges, a first waist region, a second waist region and a crotch region which extends between and connects the first waist region and the second waist region, the disposable absorbent article comprising:
a bodyfacing surface;
a garment facing surface;
an absorbent core; and
a reversible fastening system comprising:
a pair of first fasteners, at least a portion of each first fastener being situated inboard from each longitudinal side edge of the second waist region, each first fastener being configured to engage at least a portion of the garment facing surface of the first waist region; and
a pair of second fasteners, at least a portion of each second fastener being situated inboard from each longitudinal side edge of the first waist region, each second fastener being configured to engage at least a portion of the garment facing surface of the second waist region;
wherein with the lateral end edge in the first waist region and the lateral end edge in the second waist region aligned, the pair of first fasteners and the pair of second fasteners longitudinally overlap another.

2. The disposable absorbent article of claim 1 wherein the pair of first fasteners are located on a bodyfacing surface and the pair of second fasteners or located on a bodyfacing surface.

3. The disposable absorbent article of claim 1 further comprising a lateral centerline, wherein the pair of first fasteners and the pair of second fasteners are positioned substantially symmetrically about the lateral centerline.

4. The disposable absorbent article of claim 1 further comprising a lateral centerline, wherein the absorbent core is asymmetric about the lateral centerline.

5. The disposable absorbent article of claim 1 wherein the garment facing surface in the first waist region comprises a fastening panel, and the garment facing surface in the second waist region comprises a fastening panel.

6. The disposable absorbent article of claim 1 wherein the fastening panel in the first waist region comprises first indicia, the fastening panel in the second waist region comprises second indicia, the first indicia indicating a first position and the second indicia indicating a second position.

7. The disposable absorbent article of claim 6 wherein the first position is a back position and the second sleeping position is a front position.

8. The disposable absorbent article of claim 7 further comprising a lateral centerline, wherein the absorbent core has a greater absorbent capacity on the second waist region side of the lateral center line.

9. The disposable absorbent article of claim 1 further comprising a lateral centerline and a surge layer, the surge layer placed asymmetrically about the lateral centerline.

10. A disposable absorbent article having opposed longitudinal side edges, opposed lateral end edges, a lateral centerline, a first waist region, a second waist region and a crotch region which extends between and connects the first waist region and the second waist region, the disposable absorbent article comprising:
a bodyfacing surface;
a garment facing surface;
an absorbent core; and
a reversible fastening system comprising:
a pair of first fasteners, at least a portion of each first fastener being situated inboard from each longitudinal side edge of the second waist region, each first fastener being configured to engage at least a portion of the garment facing surface of the first waist region; and
a pair of second fasteners, at least a portion of each second fastener being situated inboard from each longitudinal side edge of the first waist region, each second fastener being configured to engage at least a portion of the garment facing surface of the second waist region;
wherein the first fasteners are inoperable when the second fasteners are engaged with the garment facing surface in the first waist region.

11. The disposable absorbent article of claim 10 wherein the pair of first fasteners are located on a bodyfacing surface and the pair of second fasteners or located on a bodyfacing surface.

12. The disposable absorbent article of claim 10 wherein the second fasteners are inoperable when the first fasteners are engaged with the garment facing surface in the second waist region.

13. The disposable absorbent article of claim 10 wherein the pair of first fasteners and the pair of second fasteners are positioned substantially symmetrically about the lateral centerline.

14. The disposable absorbent article of claim 10 wherein the absorbent core is asymmetric about the lateral centerline.

15. The disposable absorbent article of claim 10 wherein the garment facing surface in the first waist region comprises a fastening panel, and the garment facing surface in the second waist region comprises a fastening panel.

16. The disposable absorbent article of claim 10 wherein the fastening panel in the first waist region comprises first indicia, the fastening panel in the second waist region comprises second indicia, the first indicia indicating a first position and the second indicia indicating a second position.

17. The disposable absorbent article of claim 16 wherein the first position is a back position and the second position is a front position.

18. The disposable absorbent article of claim 17 wherein the absorbent core has a greater absorbent capacity on the second waist region side of the lateral center line.

19. The disposable absorbent article of claim 10 further comprising a surge layer, the surge layer placed asymmetrically about the lateral centerline.

20. A method of providing a system to diaper a child comprising:
providing the disposable absorbent article of claim 1;
providing instructions to a care give to don the disposable absorbent article in one of two conditions; and
enabling the wearer to successfully apply the disposable absorbent article by following the instructions.
